(19) United States
(12) Patent Application Publication
(10) Pub. No.: US 2012/0238989 A1
(43) Pub. Date: Sep. 20, 2012

(54) DISPOSABLE DIAPER

(75) Inventors: Tomomi Oku, Kagawa (JP); Satoru Sakaguchi, Kagawa (JP); Hideki Matsushima, Kagawa (JP)

(73) Assignee: UNICHARM CORPORATION, Ehime (JP)

(21) Appl. No.: 13/498,606

(22) PCT Filed: Oct. 1, 2009
(86) PCT No.: PCT/JP2010/005924

§ 371 Date: Jun. 11, 2012

(30) Foreign Application Priority Data
Oct. 1, 2009 (JP) 2009-230042

(51) Int. Cl. A61F 13/58; A61F 13/49

(52) U.S. Cl. 604/385.24; 604/389

(57) ABSTRACT

A disposable diaper includes: a main body, a side flap, and a pair of fastening tapes. The main body includes a liquid permeable topsheet, a liquid impermeable outer sheet, and an absorber between the topsheet and the outer sheet. The side flap covers one of edge portions of the main body in a longitudinal direction and has a greater dimension in a width direction than the main body. The fastening tapes are attached to respective edge portions, in the width direction, of the side flap and configured to be fastened onto a fastening portion on an opposite one of the edge portions of the main body. One of edge portions of the absorber in the longitudinal direction is located inboard of an outer imaginary line connecting outermost points of the fastening tapes in the longitudinal direction.

### TECHNICAL FIELD

The present disclosure relates to a disposable diaper.

### BACKGROUND ART

An open-type disposable diaper includes a waistline member and a main body and is provided in a state where these members are not completely joined together. The open-type diaper is formed into a pants shape by using fastening tapes to fasten the waistline member onto the main body, as disclosed, for example, in Japanese Patent Application Publication No. 2000-126231.

Generally, the open-type diaper offers a less snug fit than a pants-type diaper, which is formed into pants in advance, and may have a problem in that some postures or movements of a wearer might cause bodily waste to leak out between the diaper and the skin of the wearer.

### SUMMARY OF INVENTION

In an aspect, a disposable diaper includes: a main body, a side flap, and a pair of fastening tapes. The main body includes a liquid permeable topsheet, a liquid impermeable outer sheet, and an absorber between the topsheet and the outer sheet. The side flap covers one of edge portions of the main body in a longitudinal direction and has a greater dimension in a width direction than the main body. The fastening tapes are attached to respective edge portions, in the width direction, of the side flap and configured to be fastened onto a fastening portion on an opposite one of the edge portions of the main body. One of edge portions of the absorber in the longitudinal direction is located inboard of an outer imaginary line connecting outermost points of the fastening tapes in the longitudinal direction.

### BRIEF DESCRIPTION OF DRAWINGS

FIG. 1 is a plan view of a disposable diaper according to an embodiment of the present invention.

FIG. 2 is an exploded perspective view of the disposable diaper.

FIG. 3 is a cross-sectional view taken along the A-A′ line in FIG. 1.

FIG. 4 is a cross-sectional view taken along the B-B′ line in FIG. 1.

FIG. 5 is a plan view of the disposable diaper, illustrating joint areas between a main body and side flaps.

FIG. 6 is a plan view illustrating the positional relationship among members constituting the disposable diaper.

FIG. 7 is a plan view illustrating a fastening area F in an open state of the disposable diaper.

FIG. 8 is a partial, perspective view illustrating an in-use state of the disposable diaper as being worn.

FIG. 9 is a diagram of various steps for manufacturing the disposable diaper according to an embodiment of the present invention.

### DESCRIPTION OF EMBODIMENTS

One or more embodiment of a disposable diaper according to the present invention will be now described with reference to the drawings. Note that, in the following description of the drawings, the same or similar reference numerals denote the same or similar elements/portions. In addition, it should be noted that the drawings are schematic and are not to scale unless otherwise specified. Moreover, the drawings do not necessarily reflect the real life dimensional relationships and ratios of components.

Disposable Diaper

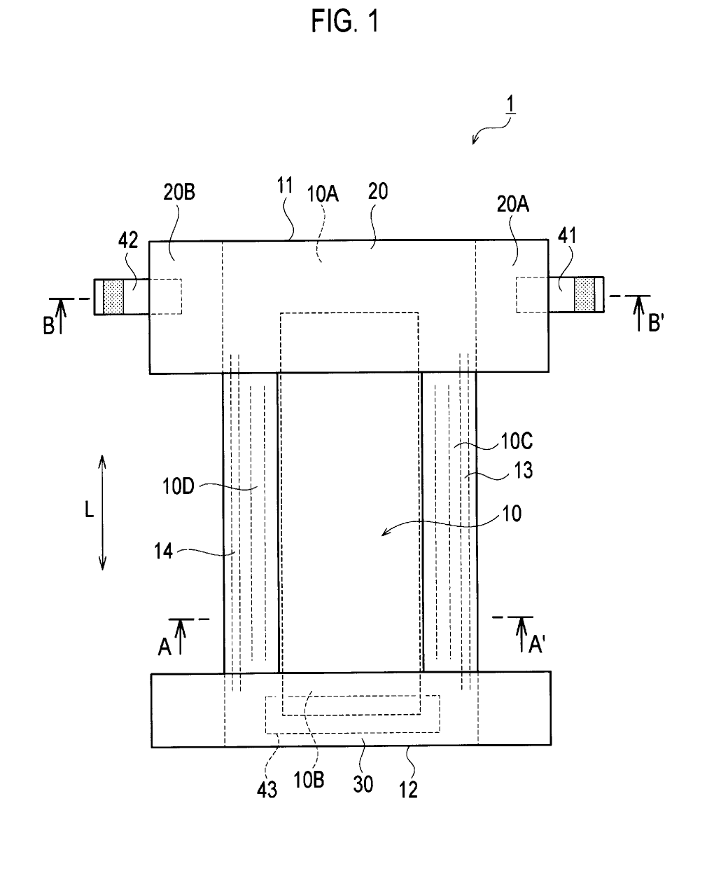

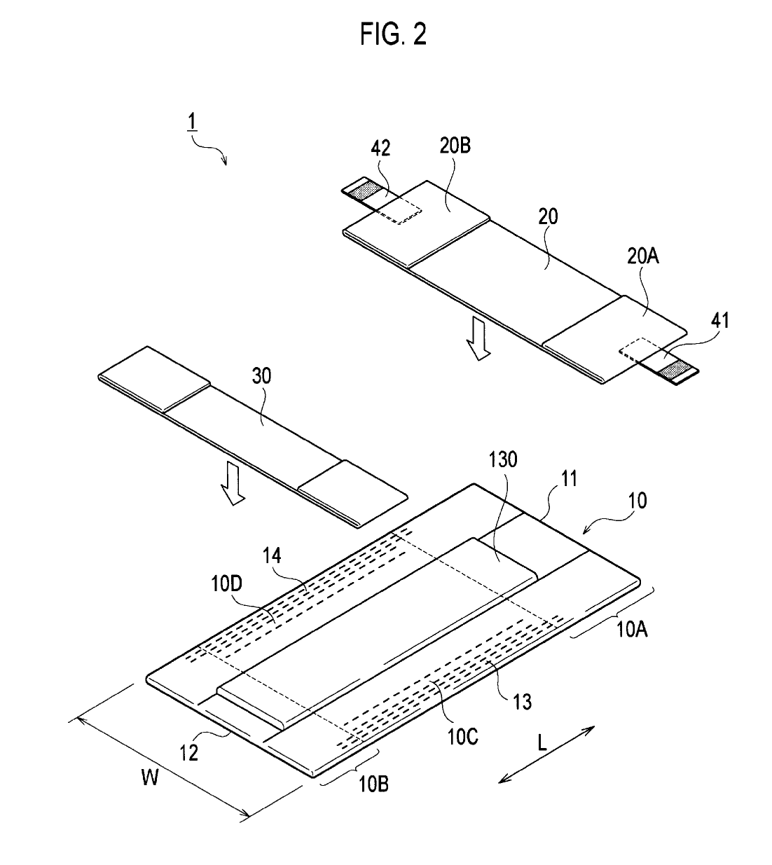

FIG. 1 is a plan view of a disposable diaper 1 according to the embodiment of the present invention. FIG. 2 is an exploded perspective view of the disposable diaper 1. The disposable diaper 1 includes a main body 10, a back side flap 20, and an abdomen side flap 30. The back side flap 20 of the disposable diaper 1 is applied to a back waistline area of a wearer. The abdomen side flap 30 is applied to an abdominal waistline area of the wearer. The main body 10 is applied to a crotch of the wearer.

The main body 10 in the specifically illustrated configuration is rectangular. The back side flap 20 is attached to an edge portion area 10A of the main body 10. The edge portion area 10A includes at least an edge portion 11 which is one of edge portions, in a longitudinal direction L, of the main body 10. The back side flap 20 is longer than a width W in a width direction of the main body 10, and covers the edge portion area 10A. The abdomen side flap 30 is attached to an edge portion area 10B of the main body 10. The edge portion area 10B includes an edge portion 12 which is the other one of the edge portions, in the longitudinal direction L, of the main body 10. The abdomen side flap 30 is longer than the width W of the main body 10, and covers the edge portion area 10B. Gathers 13 and 14 are formed in respective edge portion areas 10C and 10D, in the width direction, of the main body 10. The main body 10 and the back side flap 20 are joined together and the main body 10 and the abdomen side flap 30 are joined together by use of a joining material such as HMA (hot-melt adhesive).

The back side flap 20 and the abdomen side flap 30 are each formed of a hydrophobic nonwoven fabric, a moisture permeable or moisture impermeable film, or a compound sheet obtained by bonding together the hydrophobic nonwoven fabric and the moisture permeable or moisture impermeable film. Exemplary materials include: a film mainly formed of polyethylene, polypropylene, or the like; a breathable resin film; and a sheet obtained by joining a breathable resin film onto a nonwoven fabric such as a spunbond, a spunlace, or the like.

As an example, the back side flap 20 and the abdomen side flap 30 are each formed of an SMS nonwoven fabric (a compound nonwoven fabric formed of a melt-blown nonwoven fabric sandwiched between two layers of spun-bond nonwoven fabric) having a basis weight of 13 g/m². The back side flap 20 is doubled at least at its free ends 20A and 20B in the width direction of the main body 10.

Fastening tapes 41 and 42 are joined to the back side flap 20 by using a joining material such as HMA. The fastening tapes 41 and 42 are attached to the respective free ends 20A and 20B of the back side flap 20. The fastening tapes 41 and 42 are to be releasably fastened to the edge portion area 10B which is at the opposite side in the longitudinal direction L of the main body 10. The edge portion area 10B of the main body 10 has, at its garment-facing side, a fastening portion 43 onto which the fastening tapes 41 and 42 are to be releasably fastened. The fastening tapes 41 and 42 and the fastening portion 43 form a so-called loop-and-hook fastener in which the fastening tapes 41 and 42 are male members, and the fastening portion 43 is a female member. The reversed arrangement is within the scope of this disclosure.

The fastening tapes 41 and 42 are each formed to have an engagement surface having multiple groups of hooked projections (also referred to herein as “hooks”). Examples of the material for the fastening tapes include a polypropylene spunbond nonwoven fabric (PPSB). In some embodiments, it is preferable to use a PPSB with a basic weight of 80 g/m². The basis weight of the hooks is preferably 100 g/m² in some embodiments.

The fastening portion 43 onto which the fastening tapes 41 and 42 are to be releasably fastened is formed of a nonwoven fabric or a knitted cloth with which the hooks are releasably engageable. Compound fibers having a core-in-sheath structure are used as fibrous materials constituting the nonwoven fabric in some exemplary configurations. Examples of core-component/sheath-component compositions for the fibrous materials include polypropylene (PP)/polyethylene (PE), PP/low-melting PP, polyethylene terephthalate (PET)/low-melting PET, and PET/PE. In some embodiments, fiber such as Rayon, PET, PP, Polyamide such as nylon, acryl, urethane, cotton, or the like is mixed into the fibrous materials. The basis weight of the female member is, for example, 38 g/m².

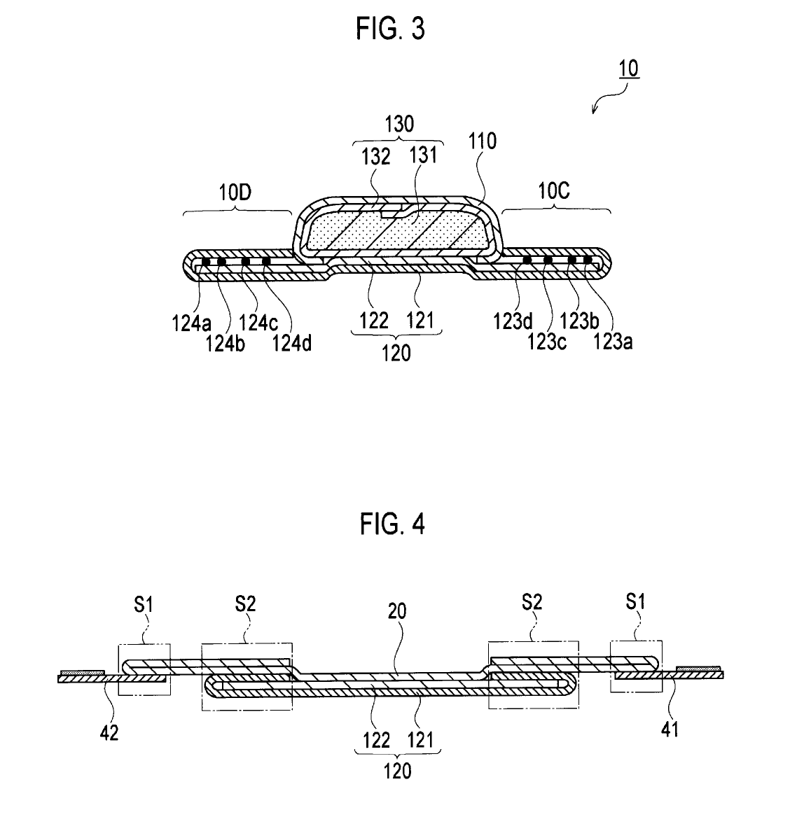

The configuration of the main body 10 will be described next. FIG. 3 is a cross-sectional view of the disposable diaper 1, taken along the A-A′ line. FIG. 4 is a cross-sectional view of the disposable diaper 1, taken along the B-B′ line.

The main body 10 includes a liquid permeable topsheet 110 arranged on a skin facing side, an outer sheet 120 placed on the garment-facing side, and an absorber 130.

The material for the topsheet 110 is not particularly limited as long as it is a sheet-like material having a liquid permeable structure, such as a nonwoven fabric, a woven fabric, a perforated plastic sheet, or a mesh sheet. Any natural fiber and/or chemical fiber is/are usable as the material for the woven fabric and/or the nonwoven fabric.

Examples of the natural fiber include cellulose such as ground pulp or cotton. Examples of the chemical fiber include regenerated cellulose such as rayon or fibrillated rayon, semi-synthetic cellulose such as acetate or triacetate, thermoplastic hydrophobic chemical fibers, or hydrophilically-treated thermoplastic hydrophobic chemical fibers. Examples of the thermoplastic hydrophobic chemical fibers include single fibers such as polyethylene (PE), polypropylene (PP), or polyethylene terephthalate (PET), fibers obtained through graft polymerization of polyethylene and polypropylene, and compound fibers having a core-in-sheath structure. For example, the topsheet 110 is an air-through nonwoven fabric with a basic weight of 18 g/m².

To form a nonwoven fabric web, any one of a dry method (such as a carding method, a spunbond method, a melt-blown method, or an air-laying method), a wet method or any combinations of multiple such dry and/or wet methods can be used. Other methods include thermal bonding, needle punching, chemical bonding, and the like. The method for forming the nonwoven fabric is not limited to the methods given above.

The outer sheet 120 includes a nonwoven fabric 121 (also referred to herein as a back nonwoven fabric) on the garment-facing side and a liquid impermeable film 122 (also referred to herein as a back film) positioned on the skin-facing side. The back film 122 is formed of a moisture permeable or moisture impermeable film. The back nonwoven fabric 121 is a hydrophobic nonwoven fabric formed of an SMS nonwoven fabric, a spunbond nonwoven fabric, or a point-bonded nonwoven fabric. The back nonwoven fabric 121 and the back film 122 are joined together using HMA or the like. For example, the back film 122 is a non-breathable film having a basis weight of 20 g/m², and the back nonwoven fabric 121 is an SMS nonwoven fabric having a basis weight of 13 g/m².

The back nonwoven fabric 121 is folded back on the back film 122 at first positions outboard of respective edges, in the width direction, of the back film 122. The back side flap 20 is folded back upon itself at second positions further outboard of the first positions in the width direction, and thus forms the free ends 20A and 20B. The fastening tapes 41 and 42 are attached to the free ends 20A and 20B, respectively.

A joining material (not shown) such as HMA is used to fixate joint portions (S1 in FIG. 4) between the back side flap 20 and the fastening tapes 41 and 42, and to fixate joint portions (S2 in FIG. 4) between the back side flap 20 and the main body 10. Accordingly, even if the material is stretched by the movement of the wearer wearing the diaper 1, the joint portions S1 and S2 do not easily stretch and thus tend to accumulate strain. In this regard, the strength of the back side flap 20 is increased at the joint portions S1 and S2, which tend to accumulate strain, by multiple layers of the back side flap 20 folded upon itself in the width direction.

In the edge portion areas 10C and 10D, in the width direction, of the main body 10, multiple elastics (elastic members) 123a, 123b, 123c, 123d, 124a, 124b, 124c, and 124d which are long and narrow (elongated) in the longitudinal direction are placed on the skin-facing side of the back film 122 while being stretched in the longitudinal direction L of the main body 10. The gathers 13 and 14 are formed by folding edge portion areas, in the width direction, of the back nonwoven fabric 121 back on those elastics.

The outer elastics 123a, 123b, 123c, 124a, 124b, and 124c that are placed in outer portions, in the width direction, of the main body 10 each have a diameter of 670 dtex, for example. These elastics are placed while being stretched 2.5 times their usual, unstretched length. In addition, the inner elastics 123d and 124d that are placed at inner positions, in the width direction, of the main body 10 each have a diameter of 470 dtex. These elastics are placed while being stretched 2.3 times their usual, unstretched length.

The absorber 130 includes an absorbent core 131 configured to absorb liquid and an absorbent sheet 132. The absorbent core 131 includes hydrophilic fibers and a high-absorbent polymer. Examples of the hydrophilic fibers include cellulose, such as ground pulp or cotton, regenerated cellulose, such as rayon or fibrillated rayon, semi-synthetic cellulose, such as acetate or triacetate, a granular polymer, a fibrous polymer, thermoplastic hydrophobic chemical fibers, hydrophilically-treated thermoplastic hydrophobic chemical fibers, or any mixture thereof. Among these materials, the ground pulp is preferably used in some embodiments, considering its low cost and workability of forming an absorber.

The absorbent core 131 is wrapped by the absorbent sheet 132. In a specific configuration of the present embodiment, the absorbent sheet 132 is a tissue. For example, a tissue having a basis weight of 15 g/m² can be used. Using HMA or the like, the absorber 130 is joined to the skin-facing side of the back film 122 while being wrapped by the topsheet 110.

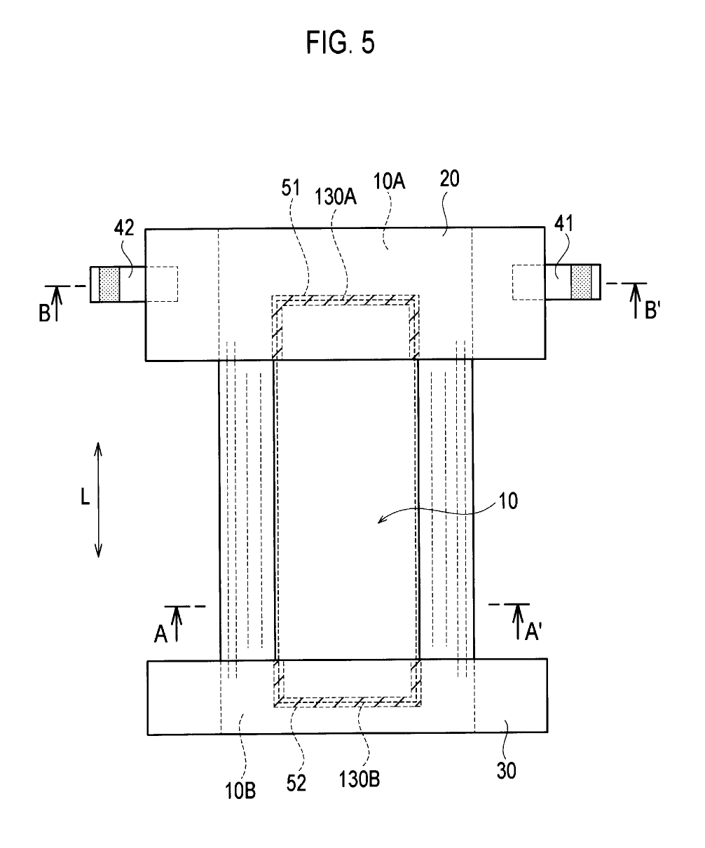

FIG. 5 is a plan view of the disposable diaper 1, illustrating a joint area between the back side flap 20 and the main body 10 and a joint area between the abdomen side flap 30 and the main body 10.

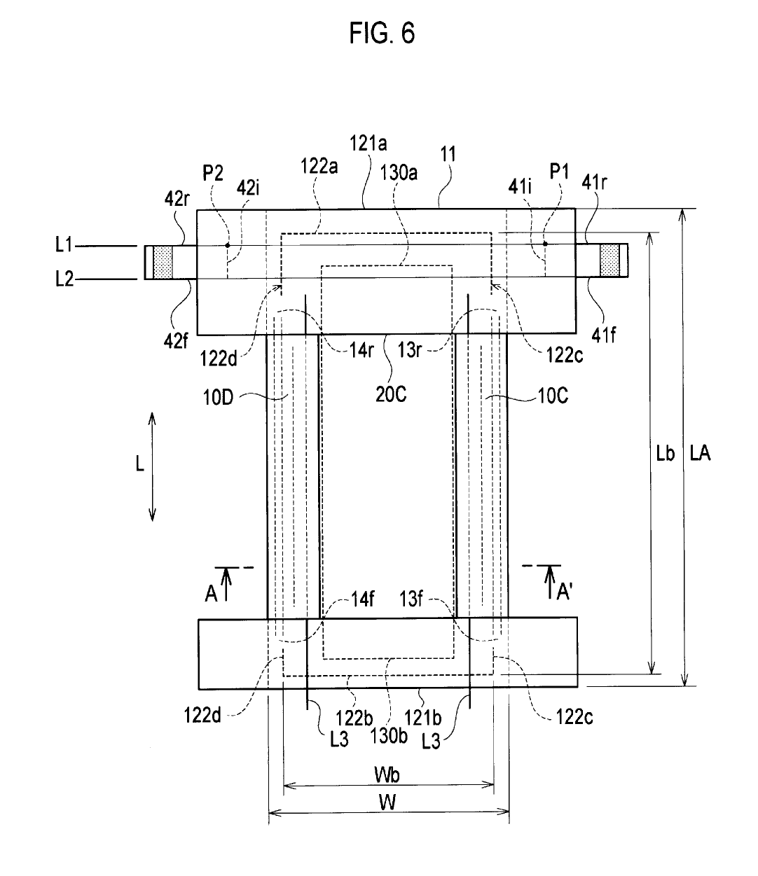

The back side flap 20 and the main body 10 are joined together at a joint area 51, and the abdomen side flap 30 and the main body 10 are joined together at a joint area 52. As shown in FIG. 5, the joint area 51 includes at least an absorber edge portion 130a (see FIG. 6) and an outer border portion 130A of the absorber 130 that are covered with the back side flap 20. The joint area 52 includes an absorber edge portion 130b (see FIG. 6) and an outer border portion 130B of the absorber 130 that are covered with the abdomen side flap 30. A joining material such as HMA can be used for the joining in the joint areas 51, 52.

The main body 10 and the back side flap 20 as well as the main body 10 and the abdomen side flap 30 may be joined together at areas other than the joint areas 51 and 52, respectively. For example, the main body 10 and the back side flap 20 may be joined together at the outer border portion 130A of the absorber 130 and an entire area outside the outer border portion 130A. Likewise, the main body 10 and the abdomen side flap 30 may be joined together at the outer border portion 130B and an entire area outside the outer border portion 130B.

However, the back side flap 20 and the main body 10 are not joined together (free of direct attachment) at an area inside the outer border portion 130A. Likewise, the abdomen side flap 30 and the main body 10 are not joined together at an area inside the outer border portion 130B.

Next, the positional relationship among the back nonwoven fabric 121, the back film 122, and the absorber 130 that form the disposable diaper 1 will be described using FIG. 6.

Imaginary lines are defined between the positions where the paired fastening tapes 41 and 42 are attached to the respective edge portions, in the width direction, of the back side flap 20. Specifically, an imaginary line is defined by connecting the fastening tapes 41 and 42 at their respective outermost or innermost points in the longitudinal direction L. In a specific configuration of the present embodiment, one imaginary line can be defined as a line connecting corners P1 and P2 where inner sides 41i and 42i (in the width direction of the main body 10) and outer sides 41r and 42r (in the longitudinal direction L of the main body 10) of the paired fastening tapes 41 and 42 intersect, respectively. Likewise, another imaginary line can be defined as a line connecting corners (unnumbered) where inner sides 41i and 42i (in the width direction of the main body 10) and inner sides 41f and 42f (in the longitudinal direction L of the main body 10) of the paired fastening tapes 41 and 42 intersect, respectively.

In a specific configuration of the present embodiment, the paired fastening tapes 41 and 42 are attached at almost symmetrical positions in the respective free ends 20A and 20B of the back side flap 20 and are almost rectangular. Accordingly, an outer imaginary line L1 is defined by connecting the outer sides 41r and 42r (in the longitudinal direction L of the main body 10) of the fastening tapes 41 and 42. Further, an inner imaginary line L2 is defined by connecting the inner sides 41f and 42f (in the longitudinal direction L of the main body 10) of the fastening tapes 41 and 42.

In a specific configuration of the present embodiment, the absorber edge portion 130a, in the longitudinal direction, of the absorber 130 is located inboard of the outer imaginary line L1 in the longitudinal direction. It is preferable in some embodiments that the absorbent core 131, the absorbent sheet 132, and the topsheet 110 have equal dimension in the longitudinal direction L. In addition, it is preferable in some embodiments that the absorber edge portion 130a of the absorber 130 be located inboard of the inner imaginary line L2 and be distanced from the inner imaginary line L2 by 50 mm or less. If the absorber edge portion 130a is distanced from the inner imaginary line L2 by more than 50 mm, a fastening force exerted by the fastening tapes 41 and 42 to the waistline is less likely to reach the absorber 130. Thereby, a force to push the absorber 130 against the crotch is insufficient, so that some movements of the wearer might sway the absorber 130 in a front-back direction of the wearer, and deform or twist the disposable diaper 1.

In a specific configuration of the present embodiment, a length Lb of the back film 122 is smaller than a length LA of the main body 10, and a width Wb of the back film 122 is smaller than a width W of the main body 10. The back film 122 extends beyond the absorber 130 under the back side flap 20.

An edge portion 122a which is one of edge portions, in the longitudinal direction, of the back film 122 is located outboard of the inner imaginary line L2 and inboard of an edge portion 121a, in the longitudinal direction, of the back nonwoven fabric 121. An edge portion 122b which is the other edge portion of the back film 122 is located inboard of an edge portion 121b, in the longitudinal direction, of the back nonwoven fabric 121. Further, under the abdomen side flap 30, the back film 122 extends beyond the absorber 130.

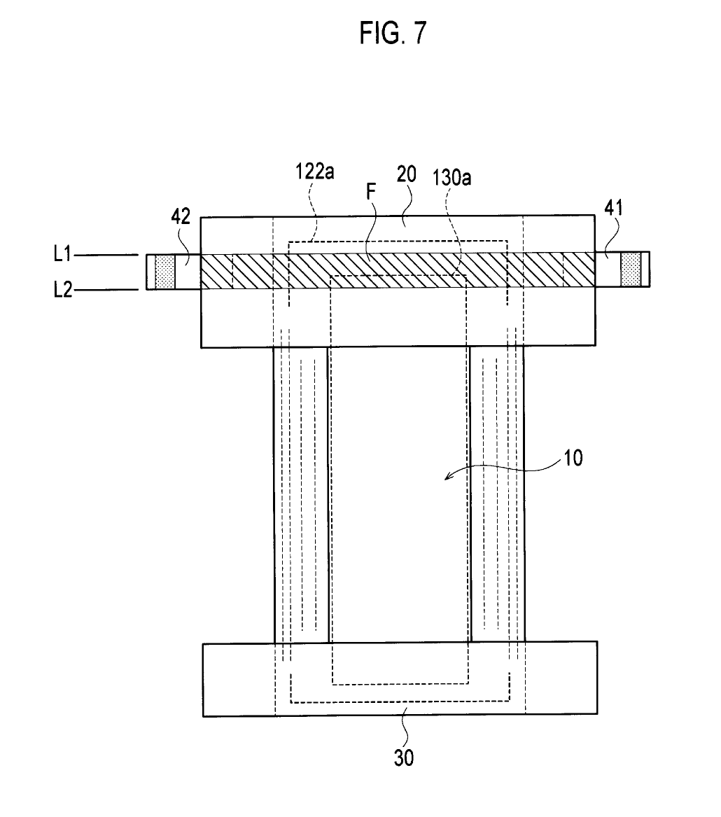

In a specific configuration of the present embodiment, the fastening portion 43 to which the fastening tapes 41, 42 are to be fastened has been described as being located in the edge portion area 10B on the garment-facing side of the main body 10. Here, it is more preferable in some embodiments that the absorber edge portion 130b does not overlap the fastening portion 43. If the absorber edge portion 130b does not overlap the fastening portion 43, the absorber 130 does not affect a fastening area (described later with respect to FIG. 7) defined by the fastening tapes 41 and 42 when the fastening tapes 41 and 42 are fastened onto the fastening portion 43 under the abdomen side flap 30. As a result, the diaper 1 can be securely fastened all around the waistline equally, and can be fit to the wearer better.

Edge portions 122c and 122d, in the width direction, of the back film 122 are located inboard of edge portions of the main body that define the width W of the main body 10, and outboard of imaginary lines L3 defined at the positions of the innermost elastics 123d and 124d, respectively.

In a plan view of the disposable diaper 1, end portions 13r and 14r of the gathers 13 and 14 overlap the back side flap 20, and end portions 13f and 14f of the gathers 13 and 14 overlap the abdomen side flap 30. Here, the end portions 13r and 13f are respective end portions of a longitudinal effective length of the gather 13, and the end portions 14r and 14f are respective end portions of a longitudinal effective length of the gather 14.

Herein, the effective length of the gathers 13, 14 is the length of each elastic, in a stretched state, fixed to the sheet forming the gather 13 or 14. In a specific configuration of the present embodiment, the effective length of the gathers 13, 14 is the length of each of the elastics 123a, 113b, 124a, and 124b. Further, the end portions of the effective length is both end portions of the elastics 123a and 123b and both end portions of the elastics 124a and 124b. In some exemplary configurations, the overlapping portions between the end portions 13r, 14r and the back side flap 20 have a length from 5 mm to 30 mm in the longitudinal direction. The overlapping portions between the end portions 13f, 14f and the abdomen side flap 30 have a length from 5 mm to 30 mm in the longitudinal direction L.

In the disposable diaper 1, the absorber end portion 130a of the absorber 130 is located inboard of the outer imaginary line L1 in the longitudinal direction L. In addition, the back side flap 20 is joined to at least the outer border portion 130A of the absorber 130.

In other words, the absorber 130 and the back side flap 20 are not joined together at an area inside the outer border portion 130A in the longitudinal direction L. When being worn, the disposable diaper 1 changes shape according to the posture of the wearer. Then, a center portion of an edge portion 20C of the back side flap 20 located above the absorber 130 often bulges away from the topsheet 110 (and toward the wearer), with the outer border portion 130A where the absorber 130 and the back side flap 20 are joined together serving as a supporting point. Thus, a space (a pocket) is formed between the back side flap 20 and the skin-facing side of the absorber 130. This space catches bodily waste. Accordingly, a leak of bodily waste toward the back can be prevented.

FIG. 7 is a plan view illustrating an area F in the back side flap 20 between the outer imaginary line L1 and the inner imaginary line L2, in an opened state of the disposable diaper 1.

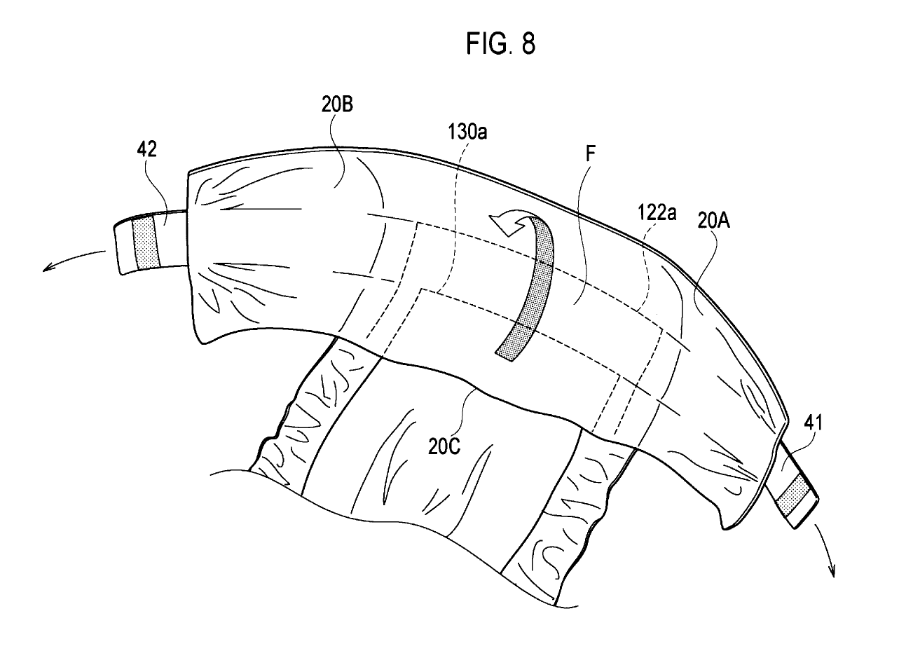

FIG. 8 is a partial, perspective view illustrating the disposable diaper 1 in an in-use state where the fastening tapes 41 and 42 are pulled outward in the width direction. When the disposable diaper 1 is being worn, the fastening tapes 41 and 42 are pulled outward along the waistline of the wearer, and the disposable diaper 1 changes shape as shown in FIG. 8.

In the disposable diaper 1, the edge portion 122a, in the longitudinal direction, of the back film 122 is located outboard of the inner imaginary line L2. In addition, the absorber edge portion 130a of the absorber 130 is located, in the longitudinal direction, inboard of the outer imaginary line L1. For that reason, a rigidity of the main body 10 differs between the position of the absorber edge portion 130a of the absorber 130 and the position of the edge portion 122a of the back film 122. By this difference in rigidity, the position of the absorber edge portion 130a of the absorber 130 and the position of the edge portion 122a of the back film 122 tend to serve as bending points.

The main body 10 is joined to the back side flap 20 on the garment-facing side of the back side flap 20. Accordingly, when the back side flap 20 is stretched outward in the width direction, the main body 10 is unlikely to receive the force resulted from of the outward stretching of the back side flap 20 in the width direction. As a result, in the area F where such force acts most intensely, the back side flap 20 and the main body 10 receive different strains from the outward stretching of the back side flap 20 in the width direction.

In the disposable diaper 1, since the position of the absorber edge portion 130a of the absorber 130 and the position of the edge portion 122a of the back film 122 tend to serve as bending points, the back side flap 20 tends to bend toward the wearer at these bending point. Accordingly, the disposable diaper 1 can bend according to the posture of the wearer.

In addition, when the disposable diaper 1 is being worn, the diaper is fit to the waistline of the wearer at the position of the absorber edge portion 130a of the absorber 130 and the position of the edge portion 122a of the back film 122 in the area F of the back side flap 20 sandwiched between the fastening tapes 41 and 42. This improves a fit of the disposable diaper 1.

It is further preferable in some embodiments that the disposable diaper 1 have a structure in which the absorber 130 does not exist in the fastening area F. When the absorber 130 does not exist in the fastening area F, the back side flap 20 can be fit continuously along the waistline of the wearer. This improves a fit of the disposable diaper 1 to the wearer's body, and accordingly can prevent a leak of bodily waste toward the back. Combined with the effect offered by the space (pocket) described earlier, a leak of bodily waste toward the back can be further prevented.

Moreover, in the disposable diaper 1, the end portion 122a of the back film 122 is located, in the longitudinal direction L, outboard of the inner imaginary line L2 and inboard of the end portion 121a of the back nonwoven fabric 121. Absorbing bodily waste, the absorber 130 increases in weight. In the disposable diaper 1, since the end portion 122a of the back film 122 is in the fastening area F or outboard of the fastening area F in the longitudinal direction, the back film 122 can support even the absorber 130 with an increased weight, and keep the absorber 130 at a position close to the skin. Moreover, since the end portion 122a of the back film 122 is located, in the longitudinal direction, outboard of the absorber edge portion 130a of the absorber 130, even if bodily waste leaks from the absorber 130, the back film 122 can prevent the bodily waste from further seeping through the back surface of the diaper.

In the disposable diaper 1, the length Lb of the back film 122 is smaller than the length LA of the main body 10. Accordingly, the edge portion areas 10A and 10B of the main body 10 each have an area where only the back side flap 20 and the back nonwoven fabric 121 are laminated. Since this area has a lower number of lamination layers than where the back film 122 is present, the disposable diaper 1 is improved in breathability.

Moreover, in the back side flap 20, there is a thickness difference between the area where only the back side flap 20 and the back nonwoven fabric 121 are laminated and the area where the back side flap 20, the back nonwoven fabric 121, and the back film 122 are laminated. This causes a difference in rigidity. Border portions of areas having a rigidity difference tend to serve as bending points. Accordingly, the disposable diaper 1 can bend naturally according to the posture of the wearer, and therefore offers an improved fit.

In addition, the width Wb of the back film 122 is smaller than the width W of the main body 10. The edge portions 122c and 122d, in the width direction, of the back film 122 are further located outboard of the imaginary lines L3 defined at the positions of the innermost elastics 123d and 124d, respectively. Accordingly, the edge portion areas 10C and 10D of the main body 10 each have an area where only the back nonwoven fabric 121 is folded. This improves the breathability of the disposable diaper 1.

In this area where only the back nonwoven fabric 121 is folded, the elastics 123a, 123b, 124a, and 124b are placed. In this area, since the number of material laminations is small, the material easily follows the stretch or contraction of the elastics. This area is particularly intended to be in contact with an edge of the crotch region of the wearer. Thus, a fit of the disposable diaper 1 improves.

In the disposable diaper 1, the elastics 123a, 123b, 123c, and 123d and the elastics 124a, 124b, 124c, and 124d are placed, in a stretched state, in the gathers 13 and 14, respectively. The end portions 13r and 14r of the effective lengths of the gathers 13 and 14 overlap the back side flap 20. Likewise, the end portions 13f and 14f of the effective lengths of the gathers 13 and 14 overlap the abdomen side flap 30.

For this reason, the portions where the back side flap 20 overlaps the gathers 13 and 14 or the portions where the abdomen side flap 30 overlaps the gathers 13 and 14 are also stretchable longitudinally of the elastics. In other words, these overlapping portions are included in the effective areas of the gathers. Since the back side flap 20 and the abdomen side flap 30 are partially stretchable longitudinally at least in the overlapping portions as described above, a gap is prevented from being formed between the skin and intersecting portions where the main body 10 covering the crotch portion meets the back side flap 20 or the abdomen side flap 30 covering the waistline.

Method for Manufacturing Disposable Diapers

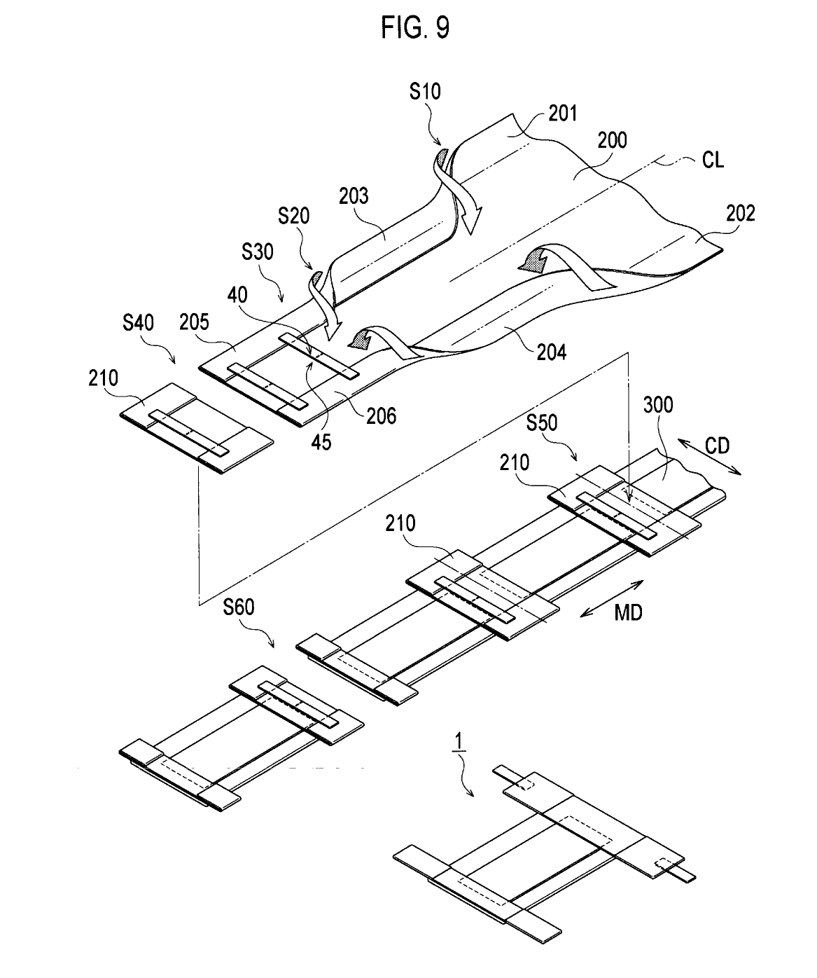

Next, a method for manufacturing the disposable diaper 1 will be described with reference to FIG. 9. As FIG. 9 shows, the method for manufacturing the disposable diaper 1 includes a first folding step S10, a second folding step S20, a fastening tape attaching step S30, a first cutting step S40, a flap attaching step S50, and a second cutting step S60.

In the first folding step S10, a flap sheet 200 continuously fed in a machine direction

MD is folded in a way that both of its side edge portions 201 and 202 are folded toward a center line CL onto an upper surface of the flap sheet 200, and the folded side edge portions 201 and 202 are then joined to the upper surface of the flap sheet 200 using HMA, embossing, or the like. The center line CL is the center of the flap sheet 200 in the width direction (or cross direction) orthogonal to the machine direction MD.

In the second folding step S20, folded edge portions 203 and 204 obtained after the first folding steps S10 are further folded toward the center line CL onto the upper surface of the flap sheet 200. Thereby, overlapping portions 205 and 206 where the flap sheet 200 is folded multiple times are formed at both edge portions of the flap sheet 200. These overlapping portions 205 and 206 constitute the free ends 20A and 20B of the back side flap 20, respectively. The overlapping portions 205 and 206 formed in the second folding step S20 are not joined to the flap sheet 200.

In the fastening tape attaching step S30, the fastening tapes 41 and 42 are attached to the flap sheet 200 at predetermined intervals in the machine direction MD. In the manufacturing process, the fastening tapes 41 and 42 are an integral member (also referred to herein as a fastening tape 40). The fastening tape 40 is processed, at its center, to have a perforation line 45 or the like in the machine direction MD that facilitates separation.

In the first cutting step S40, the flap sheet 200 is cut in the cross direction CD orthogonal to the machine direction MD at predetermined intervals. Each piece 210 cut from the flap sheet 200 defines one back side flap 20 and one abdomen side flap 30 which are integrated with each other.

In the flap attaching step S50, the cut pieces 210 cut from the flap sheet 200 in the step S40 are placed on a main body sheet 300 at predetermined intervals. The main body sheet 300 is formed by placing absorbent cores intermittently on a continuous web of outer sheets. Each cut piece 210 is placed on the main body sheet 300 to cover a gap between the intermittently placed absorbent cores, and is joined at a predetermined joint area which in some embodiments includes at least the area 130A disclosed with respect to FIG. 5.

In the second cutting step S60, the main body sheet 300 onto which the cut pieces 210 are attached is cut in the cross direction CD. Each cut piece 210 is cut into the abdomen side flap 30 of one diaper and the back side flap 20 of another diaper. The step S60 completes the disposable diaper 1.

### Other Embodiments

As described above, the details of several embodiments of the present invention have been exemplarily disclosed. However, it should not be understood that the description and drawings which constitute part of this disclosure limit the present invention. From this disclosure, various alternative embodiments, examples, and operation techniques will be found by those ordinarily skilled in the art.

For examples, the description has been given for the disposable diaper 1 in which the back side flap 20 and the abdomen side flap 30 are attached to the main body 10. However, further embodiments are not limited thereto. For example, some embodiments of the present invention may be applied to a pants-type disposable diaper in which a waistline area for the waistline of a wearer and a crotch area for the crotch portion are integrally formed and in which openings are formed for the legs of the wearer.

The disposable diaper 1 may be for infants, toddlers, or adults.

Note that the entire contents of the Japanese Patent Application No. 2009-230042 filed on Oct. 1, 2009 is incorporated herein by reference.

Further, the aspects of the present invention may be arranged described in at least the following items. In the disposable diaper, said one of the edge portions of the absorber in the longitudinal direction may be located inboard of an inner imaginary line that connects inner sides of the fastening tapes in the longitudinal direction.

In the disposable diaper, said one of the edge portions of the absorber in the longitudinal direction may be spaced from the inner imaginary line by 50 mm or less in the longitudinal direction.

In the disposable diaper, the outer sheet may include a liquid impermeable back film placed on a skin-facing side adjacent the absorber, and one of edge portions of the back film in the longitudinal direction may be located outboard of an inner imaginary line that connects inner sides of the fastening tapes in the longitudinal direction.

In the disposable diaper, said one of the edge portions of the back film in the longitudinal direction may be located outboard of the outer imaginary line. In the disposable diaper, said one of the edge portions of the back film in the longitudinal direction may be located between the outer and inner imaginary lines.

In the disposable diaper, the other one of the edge portions of the back film in the longitudinal direction may not overlap the fastening portion.

In the disposable diaper, the side flap may be joined to the main body in an outer border portion of the absorber along said one of the edge portions of the absorber in the longitudinal direction. In the disposable diaper, the side flap may be further joined to the main body outside the outer border portion of the absorber. In the disposable diaper, the side flap may be free of direct attachment to the main body inside the outer border portion of the absorber.

In the disposable diaper, the side flap may be joined to a skin-facing side of the main body.

The disposable diaper may comprise a further side flap covering the other one of the edge portions of the main body in the longitudinal direction and having a greater dimension in the width direction than the main body. In the disposable diaper, the further side flap may be joined to the main body in a further outer border portion of the absorber along the other one of the edge portions of the absorber in the longitudinal direction. In the disposable diaper, the further side flap may be joined to a skin-facing side of the main body.

In the disposable diaper, both edge portions of the main body in the width direction each may have a gather including a plurality of elongated elastic members fixed to the gathers in a state being stretched in the longitudinal direction, and in a plan view of the main body, the further side flap may overlap end portions of the respective elastic members in the longitudinal direction.

In the disposable diaper, the topsheet may cover at least a skin-side surface of the absorber and has substantially the same dimensions as the absorber in both the longitudinal and width directions, wherein the absorber is provided on a skin-side surface of the outer sheet.

In the disposable diaper, the absorber may have a longitudinal end portion, which includes the transverse edge of the absorber and longitudinal end portions of longitudinal edges of the absorber, which is overlapped by the side flap and sandwiched between the outer sheet and the side flap, and wherein the side flap may be joined to the absorber along the transverse edge of the absorber and along the longitudinal edges of the absorber only.

In the disposable diaper, the back film may be smaller than the back nonwoven fabric in the longitudinal and/or width directions.

In the disposable diaper, the transverse edge of the back film may be located outside, in the longitudinal direction, of the outer hypothetical line. In the disposable diaper, the transverse edge of the absorber may be located inside, in the longitudinal direction, of the inner hypothetical line by a distance of 50 mm or less.

In the disposable diaper, the transverse edge of the absorber may be located outside, in the longitudinal direction, of the inner hypothetical line. In the disposable diaper, the length of the overlap may be between 5 and 30 mm.

In the disposable diaper, a fastening portion, to which the fastening tapes are arranged to be attached, may be provided on a clothing-contact-surface side of the opposed longitudinal end portion of the main body, and wherein the opposed transverse edge of the absorber may be located inside of the fastening portion in the longitudinal direction.

1. a main body having a longitudinal direction and a width direction orthogonal to the longitudinal direction, said main body including a liquid permeable topsheet a liquid impermeable outer sheet, and an absorber between the topsheet and the outer sheet;
a side flap covering one of edge portions of the main body in the longitudinal direction and having a greater dimension in the width direction than the main body; and
a pair of fastening tapes attached to respective edge portions, in the width direction, of the side flap and configured to be fastened onto a fastening portion on an opposite one of the edge portions of the main body,
wherein
one of edge portions of the absorber in the longitudinal direction is located inboard of an outer imaginary line connecting outermost points of the fastening tapes in the longitudinal direction.

2. The disposable diaper according to claim 1, wherein
the outer sheet includes a back nonwoven fabric placed on a garment-facing side, and a liquid impermeable back film placed on a skin-facing side and between the back nonwoven fabric and the absorber, and one of edge portions of the back film in the longitudinal direction is located outboard of an inner imaginary line that connects inner sides of the fastening tapes in the longitudinal direction, and inboard of an edge portion of the back nonwoven fabric in the longitudinal direction.

3. The disposable diaper according to claim 2, wherein
both edge portions of the main body in the width direction each have a gather including a plurality of elastic members elongated in the longitudinal direction, and
edge portions of the back film in the width direction are located inboard of both the edge portions of the main body in the width direction, and outboard of innermost elastic members among the plurality of elastic members.

4. The disposable diaper according to claim 1, wherein
both edge portions of the main body in the width direction each have a gather including a plurality of linear elastic members fixed to the gathers in a state being stretched in the longitudinal direction, and in a plan view of the main body, the side flap overlaps end portions of the respective elastic members in the longitudinal direction.
